(19) United States
(12) Patent Application Publication
(10) Pub. No.: US 2014/0349923 A1
(43) Pub. Date: Nov. 27, 2014

(54) Compositions And Methods For Treating Diabetes And/Or Obesity

(71) Applicant: GlaxoSmithKline Intellectual Property (No. 2) Limited, Brentford Middlesex (GB)

(72) Inventors: Mark Andrew Paulik, Research Triangle Park, NC (US); Rebecca Jane Hodge, Research Triangle Park, NC (US)

(21) Appl. No.: 14/370,986

(22) PCT Filed: Jan. 14, 2013
(86) PCT No.: PCT/US13/21368

§ 371 Date: Jul. 8, 2014

Related U.S. Application Data
(60) Provisional application No. 61/586,929, filed on Jan. 16, 2012.

(51) Int. Cl. A61K 38/26; A61K 31/155; A61K 31/353; A61K 31/201; A61K 31/732; A61K 31/702

(52) U.S. Cl. CPC A61K 38/26; A61K 31/732; A61K 31/702; A61K 31/353; A61K 31/201; A61K 31/155
USPC 514/5.3; 514/54; 514/61; 514/7.2

(57) ABSTRACT

Compositions are disclosed which comprise one or more of the following; an anthocyanin, an oligosaccharide, a pectin, or a long-chain fatty acid. Such compositions are useful for treating diabetes or obesity.

### FIELD OF THE INVENTION

This invention relates to compositions and methods for treating diabetes or obesity. In particular, this invention relates to compositions containing one or more of the following; an anthocyanin, an oligosaccharide, a pectin, or a long-chain fatty acid.

### BACKGROUND OF THE INVENTION

Anthocyanins are molecules in the flavonoid class which are water-soluble and generally pigmented red, purple, or blue. They are found in the leaves, stems, roots, flowers, and fruits of most plants, with particularly high concentrations in plants such as blueberry, cranberry, raspberry, blackcurrant (cassis), blackberry, bilberry, purple corn, and the Amazonian palmberry (acai). Anthocyanins are strong antioxidants in vitro, but there is some evidence that they have little or no direct antioxidant effect once eaten (Lotito S B, Frei B, Consumption of flavonoid-rich foods and increased plasma antioxidant capacity in humans: Cause, consequence, or epiphenomenon?, Free Radical Biology & Medicine 41 2006 1727-1746). Studies using fruit extracts or specific anthocyanins, such as C3G (cyanidin-3-O-glucoside) have demonstrated a range of metabolic effects. In mice, C3G, and C3G-rich purple corn color have been shown to improve insulin sensitivity and reduce fasting glucose levels in high-fat and diabetic models, as well as improving levels of inflammatory cytokines and reducing hepatic triglyceride content and steatosis. See for example: Guo H, et al, Cyanidin 3-glucoside attenuates obesity-associated insulin resistance and hepatic steatosis in high-fat diet-fed and db/db mice via the transcription factor FoxO1, Journal of Nutritional Biochemistry, in press, available on line 2 May 2011; Sasaki R, et al, Cyanidin 3-glucoside ameliorates hyperglycemia and insulin sensitivity due to downregulation of retinol binding protein 4 expression in diabetic mice, Biochemical Pharmacology 2007 74:1619-27; and Tsuda T, Horio F, Uchida K, Aoki H, Osawa T, Dietary Cyanidin 3-O-D-Glucoside-Rich Purple Corn Color Prevents Obesity and Ameliorates Hyperglycemia in Mice, J. Nutr. 2003 133: 2125-2130. Anthocyanins have been proposed as effective anti-obesity agents (Tsuda 2008), as well as potential anti-cancer agents (Wang L, Stoner G D, Anthocyanins and their role in cancer prevention, Cancer Lett. 2008 Oct. 8; 269(2): 281-290). In vitro and in vivo studies have shown that anthocyanins have the ability to activate AMPK in fat cells, and to induce adiponectin gene expression through a PPARγ-independent mechanism (Tsuda T, Regulation of Adipocyte Function by Anthocyanins; Possibility of Preventing the Metabolic Syndrome, J. Agric. Food Chem. 2008, 56, 642-646), which implies they and/or their metabolites may have a direct effect on fat metabolism.

Oligosaccharides are chains of simple sugars, usually consisting of two to ten simple sugar units. Polysaccharides generally contain a greater number of simple sugars. Molecules in this category are characterized by the fact that they are not digestible in the proximal mammalian gut, but are instead partially or completely fermented by endogenous gut bacteria. This category includes, but is not limited to, the following examples. Inulin, a non-digestible, fermentable, soluble polysaccharide fiber consisting of chains of d-fructose molecules connected by β2-1 bonds with a terminal α1-2 linked d-glucose. Inulin chain length is highly variable and can range from 10 to 60 fructose molecules (a “Degree of Polymerization”, or DP, of 10 to 60). Inulin is found in a wide range of plants, including Jerusalem artichokes, chicory, onions, garlic, and asparagus. Oligofructose (OFS) is inulin that has been further hydrolyzed to produce a mixture of medium- and short-chain molecules. In some instances, molecules that are enzymatically synthesized from smaller sugar molecules to form short or medium chains are also referred to as OFS. Fructo-oligosaccharide (FOS) is a term that generally refers to even shorter fructose-chain molecules, although it is sometimes used interchangeably with OFS. These shorter FOS molecules may be found naturally in plants, or may be enzymatically synthesized from smaller sugars. In synthesized FOS molecules, bonds other than the β2-1 may exist in variable numbers. Examples of FOS include, but are not limited to, the following: Kestose (GF2), a trisaccharide polymer of two d-fructose molecules, terminated by a d-glucose molecule; Nystose (GF3), a tetrasaccharide polymer of three d-fructose molecules, terminated by a d-glucose molecule; Fructosyl nystose (GF4), a polymer consisting of 4 d-fructose molecules, terminated by a d-glucose molecule; 1β-furanosyl nystose, a 4-fructose polymer in which the terminal fructose is in the furanosyl form; Bifurcose (GF3) 1&6-kestotetraose; and Inulobiose (F2), Inulotriose (F3), and Inulotetraose (F4). Galacto-oligosaccharides (GOS), also known as oligogalactosyllactose, oligogalactose, oligolactose or transgalactooligosacchariden (TOS), are fibers consisting of chains of galactose units, with a terminal glucose. They are generally formed through enzymatic conversion of lactose, and the DP ranges from 2 to about 8. Stachyose—an oligosaccharide found in many vegetables and commonly extracted from soybeans. It is a tetrasaccharide consisting of two alpha-d-galactose units, one alpha-d-glucose unit, and one beta-d-frucose unit. Raffinose—a trisaccharide consisting of galactose, fructose, and glucose. Verbascose-6-D-Fructofuranosyl O-α-D-galactopyranosyl-(1→6)-[O-α-D-galactopyranosyl-(1→6)]2-α-D-glucopyranoside. Lactulose—a synthetic disaccharide consisting of one molecule of fructose and one of galactose. Lactosucrose—4G-beta-D-glactosylsucrose. Malto-oligosaccharides—oligosaccharides containing only alpha-1-4 glucosidic linkages. Isomalto-oligosaccharides, or branched-oligosaccharides—contain a mix of alpha 1-4 and alpha-1-6 glucosidic linkages. Xylo-oligosaccharides, agaro-oligosaccharides, manno-oligosaccharides, chitin/chitosan oligosaccharides—oligosaccharides derived from xylan, agar, mannan, chitin, and chitosan, respectively. Gentio-oligosaccharides—glucose polymers consisting of glucose units connected with beta 1-6 bonds, generally 2 to 5 units in length. Cyclo-dextrin—cyclic alpha-1,4 linked malto-oligosaccharides containing 6 to 12 glucose units.

Oligosaccharides and inulin are considered to be prebiotics, or substances that promote the growth of beneficial bacteria in the gut, particularly Bifidobacteria and Lactobacillus species. See, for example: Delzenne N M, Oligosaccharides: State of the Art, Proceedings of the Nutrition Society 2003, 62, 177-182; Ramirez-Farias C, et al, Effect of inulin on the human gut microbiota: stimulation of Bifidobacterium adolescentis and Faecalibacterium prausnitzii, British Journal of Nutrition (2009), 101, 541-550; and Niness K R, Inulin and oligofructose: What are they?, J. Nutr. 1999 129: 1402S-1406S. Because they are resistant to digestion by saliva and digestive enzymes, prebiotics pass relatively intact into the distal ileum and colon, where they are digested by endogenous bacteria. See, for example Bouhnik, Y., Raskine, L., Simoneau, G., Vicaut, E., Neut, C., Flourié, B., Brouns, F., Bornet, F. R., The capacity of nondigestible carbohydrates to stimulate fecal bifidobacteria in healthy humans: a double-blind, randomised, placebo-controlled, parallel-group, dose-response relation study, Am. J. Clin. Nutr. 2004 80:1658-64. Bacteria like Bifidobacteria and Lactobacillus digest their food intracellularly, and so are dependent on short- and medium-length fibers. Longer chain fibers pass more distally into the gut and may be digested by bacteria that excrete enzymes to permit extracellular digestion. This helps to explain different effects seen with the administration of short-chain vs. long-chain forms of these fibers.

The effects of modifying the gut flora to favor beneficial bacteria are numerous. Short-chain fatty acid production increases, the number of L-cells in the gut increases (Cani P D, Hoste S, Guiot Y, Delzenne N M, Dietary non-digestible carbohydrates promote L-cell differentiation in the proximal colon of rats, British Journal of Nutrition (2007), 98, 32-37), and, as a result, there is an increase in the release of several gut peptides, including GLP-1, GLP-2 (glucagon-like peptides 1 and 2) and PYY (peptide tyrosine tyrosine) (Delzenne N M, Cani P D, Daubioul C, Neyrinck A M, Impact of inulin and oligofructose on gastrointestinal peptides, British Journal of Nutrition (2005), 93, Suppl. 1, S157-S161). Other health-promoting benefits that have been identified include stimulation of immune function, improved absorption of nutrients such as magnesium and calcium (Van den Heuvel, E GHM, Muys T, van Dokkum W, Schaafsma G, Oligofructose stimulates calcium absorption in adolescents, Am J Clin Nutr 1999; 69:544-8), with resulting improvements in bone density, and inhibition of the growth of harmful bacteria. The GLP-2 stimulated by OFS has been shown in animal models to reduce whole-body inflammation and, specifically, inflammation in the liver (Cani P D, et al., Changes in gut microbiota control inflammation in obese mice through a mechanism involving GLP-2-driven improvement of gut permeability, Gut 2009; 58:1091-1103). Rats placed on life-long diets of OFS-enriched inulin showed improvements in general health and lifespan (Rozan P, et al, Effects of lifelong intervention with an oligofructose-enriched inulin in rats on general health and lifespan, British Journal of Nutrition (2008), 100, 1192-1199).

The effects of these fibers have been extensively studied in man (Loo J V, et al, Functional Food Properties of Non-Digestible Oligosaccharides: A Consensus Report from the ENDO project (DGXII AIRII-CT94-1095), British Journal of Nutrition 1999, 81, 121-132) for a wide range of indications, including, but not limited to: as potential therapy for Irritable Bowel Disease (IBS), Inflammatory Bowel Disease, Ulcerative Colitis, and Crohn's Disease (see, for example: Hedin, C, Whelan K, Lindsay J O, Evidence for the use of Probiotics and Prebiotics in Inflammatory Bowel Disease: A Review of Clinical Trials, Proceedings of the Nutrition Society 2007, 66, 307-315; and Leenen C H, Dielman L A, Inulin and oligofructose in chronic inflammatory bowel disease, J. Nutr. 2007 137: 2572S-2575S), for their properties of enhancing immune function, particularly in infants and in the elderly, (see, for example: Vulevic J, Drakoularakou A, Yaqoob P, Tzortzis G and Gibson G R; Modulation of the fecal microflora profile and immune function by a novel trans-galactooligosaccharide mixture (B-GOS) in healthy elderly volunteers, Am J of CI Nutr 2008 88; 1438-1446; Gibson, G. R., McCartney, A. L., Rastall, R. A., Prebiotics and resistance to gastrointestinal infections, Br J of Nutr. 2005 93, Suppl. 1, pp 31-34; and Lomax, A R, Calder, P C, Prebiotics, immune function, infection and inflammation: a review of the evidence, British Journal of Nutrition 2009, 101, 633-658), as an aid to weight loss (see, for example, Cani P D, Joly E, Horsmans Y, Delzenne N M, Oligofructose promotes satiety in healthy human: a pilot study, European Journal of Clinical Nutrition 2006 60, 567-572; and Parnell J A, Reimer R A, Weight loss during oligofructose supplementation is associated with decreased ghrelin and increased peptide YY in overweight and obese adults, Am J Clin Nutr 2009; 89:1751-9), and as a modulator of blood sugar levels in patients with diabetes (see, for example, Luo J, Yperselle M V, Rizkalla S W, Rossi F, Bornet F R J, Slama G, Chronic consumption of short-chain fructooligosaccharides does not affect basal hepatic glucose production of insulin resistance in type 2 diabetics, J. Nutr. 2000 130: 1572-1577). Results of studies have been somewhat mixed, but interest in the use of these fibers remains strong. As a result of their beneficial effects, fibers of this type are currently used to supplement a wide variety of high-fiber food products, infant formulas, and pet foods, and are combined with probiotics to improve gut health.

Few studies have been conducted in other non-rodent species, but in general, effects appear to translate well into other monogastric mammals, including dogs and cats. See, for example: Massimino S P, et al, Fermentable dietary fiber increases GLP-1 secretion and improves glucose homeostasis despite increased intestinal glucose transport capacity in healthy dogs, J. Nutr. 1998 128: 1786-1793; Bosch G, et al., The effects of dietary fibre type on satiety-related hormones and voluntary food intake in dogs, British Journal of Nutrition 2009, 102, 318-325; Respondek F, et. al., Short-chain fructooligosaccharides influence insulin sensitivity and gene expression of fat tissue in obese dogs, J. Nutr. 2008 138: 1712-1718; and Verbrugghe A, et. al., Oligofructose and inulin modulate glucose and amino acid metabolism through propionate production in normal-weight and obese cats, British Journal of Nutrition (2009), 102, 694-702.

Pectin is a complex polysaccharide found in the cell-walls of almost all terrestrial plants. Its exact structure varies according to the specific plant, the part of the plant, and its stage of its development, and components of pectin also vary according the extraction process used. It is a soluble dietary fiber which is poorly digested in the proximal digestive tract and is partially fermented by gut bacteria in the distal gut. Commercial pectins are available from many sources, but the main raw ingredients tend to be citrus peel or apple pomace. Pectin is primarily used as a gelling and thickening agent in foods such as jams, jellies, and marmalades. It has been used to increase stool viscosity and, in the past, was a key ingredient of Kaopectate. Pectin has been shown to cause significant delays in gastric emptying and improvements in satiety and has therefore been proposed as an anti-obesity agent. See, for example: DiLorenzo C, Williams C M, Hajnal F, Valenzuela J E, Pectin delays Gastric Emptying and Increases Satiety in Obese Subjects, Gastroenterology 1988 Vol 95, no 5 p 1211-1215; Sanaka M, et al, Effects of agar and pectin on gastric emptying and post-prandial glycaemic profiles in healthy human volunteers, Clinical and Experimental Pharmacology and Physiology 2007 34, 1151-1155; Schwartz S E et al, Sustained pectin ingestion: effect on gastric emptying and glucose tolerance in non-insulin-dependent diabetic patients, Am J Clin Nutr 1988; 48:1413-7; and Tiwary C M, Ward J A, Jackson B A, Effect of Pectin on Satiety in Healthy US Army Adults, Journal of the American College of Nutrition, 1997 Vol 16, No 5, 423-428. Published data are mixed, however, with some studies showing no beneficial effects on weight. See, for example, Howarth N C et al, Fermentable and Nonfermentable Fiber Supplements Did Not Alter Hunger, Satiety or Body Weight in a Pilot Study of Men and Women Consuming Self-Selected Diets, J. Nutr. 2003 133: 3141-3144. In healthy volunteers, pectin has been shown to significantly reduce blood glucose levels (Holt S, Heading R C, Carter D C, Prescott L F, Tothill P, Effect of gel fibre on gastric emptying and absorption of glucose and paracetamol, The Lancet, Mar. 24, 1979, 636-639), but some studies in subjects with type 2 diabetes have not shown glucose effects (Schwartz et al).

Long-chain fatty acids are carboxylic acids containing 12 to 22 carbon atoms, which have varying degrees of saturation. This category includes, but is not limited to: oleic acid—a monounsaturated 18-carbon carboxylic acid with a single cis double bond, found in a range of plant and animal products; Linoleic acid—a polyunsaturated 18-carbon carboxylic acid with two cis double bonds; eicosapentaenoic acid (EPA, icosapentaenoic acid, timnodonic acid)—an omega-3 fatty acid with 20 carbon atoms and three cis double bonds, found in algae and fish products; and docosahexaenoic acid (DHA, cervonic acid)—an omega-3 fatty acid with 22 carbon atoms and six cis double bonds, found in algae and fish products. Oleic acid has been shown to directly stimulate the release of multiple gut peptides in animals and in man, including GLP-1, PYY, GIP, and oxyntomodulin and, as a primary component of olive oil, has been credited with some of the positive effects seen with the Mediterranean diet, including reductions in blood pressure. See, for example: Anini Y, et al. Comparison of the postprandial release of peptide YY and proglucagon-derived peptides in the rat, Eur J Physiol 1999 438:299-306; Carr R D, et al., Incretin and islet hormonal responses to fat and protein ingestion in healthy men, Am J Physiol Endocrinol Metab 2008 295: E779-E784; and Teres S, et. al., Oleic acid content is responsible for the reduction in blood pressure induced by olive oil, PNAS 2008 105(37) 13811-13816. Linoleic acid has also been shown to stimulate GLP-1 release (Adachi T, et al, Free fatty acids administered into the colon promote the secretion of glucagon-like peptide-1 and insulin, Biochemical and Biophysical Research Communications 2006 340 332-337). In mice, supplementation with conjugated linoleic acid has been shown to reduce body fat, but studies performed in man to date have not shown consistent effects (Terpstra A H M, Effect of conjugated linoleic acid on body composition and plasma lipids in humans: an overview of the literature, Am J Clin Nutr 2004; 79: 352-61).

GLP-1 is an incretin secreted by intestinal L-cells in response to the ingestion of food. It is secreted as a thirty-amino-acid hormone (GLP-1₇₋₃₆, ‘active’ GLP-1) which is then cleaved by the enzyme dipeptidyl peptidase IV (DPP-IV) to its ‘inactive’ form, GLP-1₉₋₃₆. The active peptide plays an important role in the regulation of postprandial blood glucose levels by stimulating glucose-dependent secretion of insulin, resulting in increased glucose disposal into tissues. GLP-1 also suppresses glucagon secretion, leading to reduced hepatic glucose output. In addition, GLP-1 delays gastric emptying time and slows small bowel motility, delaying food absorption. Exendin-4, a 39-amino-acid peptide, was originally identified in the saliva of the Gila monster, Heloderma suspectum, and functions as a potent GLP-1 mimetic. (Neary M T, Batterham R I, Gut Hormones: Implications for the Treatment of Obesity, Pharmacology & Therapeutics 124 44-56 2009).

Two GLP-1 mimetics are currently approved for the treatment of type 2 diabetes melitis: exenatide (exendin-4, BYETTA, BYDUREON), and liraglutide (VICTOZA). Both agents cause weight loss in patients with diabetes, and liraglutide is also being explored as an agent for weight loss in obese non-diabetic patients. See, for example, Astrup A, et al, Effects of Liraglutide in the Treatment of Obesity: A Randomised, Double-Blind, Placebo-Controlled Study, Lancet 374:1606-16, 2009. A number of other compounds in this class are under development, including albiglutide, lixisenatide, LY2189265 (dulaglutide), PF-4856883, ZYD-1, HM11260C (LAPS Exendin), and others. In addition, several agents are under development which have activity at the GLP-1 receptor as well as at other receptor sites, including MAR-701 (GLP-1 and GIP agonist), OAP-189, ZP2929, and DualAG (GLP-1 and glucagon agonists), and ZP3022 (GLP-1 and gastrin agonist).

A number of drugs, both marketed and under development, have mechanisms that result in an increase in GLP-1 plasma concentrations. Some examples of these types of drugs include the following. Metformin is a marketed antidiabetic agent, which has been shown to increase circulating levels of GLP-1. See, for example, Maida A, Lamont B J, CaoX, Drucker D J, Metformin regulates the incretin receptor axis via a pathway dependent on peroxisome proliferator-activated receptor-α in mice, Diabetologia 2011 54(2) 339-349 and Manucci E, et al, Effect of Metformin on Glucagon-Like Peptide 1 (GLP-1) and Leptin Levels in Obese Nondiabetic Subjects, Diabetes Care 2001 24:489-494. DPP-IV inhibitors are a class of drugs which includes sitagliptin and saxagliptin as currently marketed agents, with numerous other molecules under development. Molecules in the DPP-IV class inhibit the action of the DPP-IV enzyme, thereby increasing circulating levels of active GLP-1. Bile acid sequestrants are a class of drugs which prevents re-absorption of bile acids from the intestinal tract, and which have been shown to increase GLP-1 levels. See, for example, Shang Q, Saumoy M, Holst J J, Salen G, Xu G R Colesevelam improves insulin resistance in a diet-induced obesity (F-DIO) rat model by increasing the release of GLP-1 American Journal of Physiology-Gastrointestinal and Liver Physiology 2010-298(3): G419-G424. Marketed bile acid sequestrants include colestipol, cholestryramine and colesevelam. Ileal Bile Acid Transport (iBAT) Inhibitors are drugs which prevent re-absorption of bile acids by interfering with the active transport system that moves bile acids across the gut wall. IBAT compounds currently under development include ALBI-3309, AZD-7806, S-8921, and SAR-58304 SGLT-1 Inhibitors are drugs which inhibit the SGLT-1 enzyme, which transports glucose out of the gut lumen. As unabsorbed glucose moves into the distal gut, it stimulates an increased release of GLP-1. SGLT-1 compounds currently under development include DSP-3235 (GSK1614235) and LX-4211. Agonism at the TGR5, GPR39 or GPR40 receptors can cause an increase in GLP-1. Muscarinic agonists directly stimulate the release of GLP-1. See, for example, Anini Y, and Brubaker P L, Muscarinic receptors control glucagon-like peptide 1 secretion by human endocrine L cells, Endocrinology. 2003 July; 144(7):3244-50. Muscarinic antagonists have also been shown to increase GLP-1 levels.

In a discussion of the role of the gut micorbiota on lipid metabolism, Fava F, et al. The Gut Microbiota and Lipid Metabolism: Implications for Human Health and Coronary Heart Disease, Current Medicinal Chemistry, 2006, 13, 3005-3021 reviews the cardiovascular benefits seen with administration of OFS, as well as the potential benefits of flavonoids in the diet. No experimental work describing co-administration is described. The authors summarize in part by saying that “understanding these microbial activities is central to determining the role of different dietary components in preventing or beneficially impacting on the impaired lipid metabolism and vascular dysfunction that typifies CHD and type II diabetes. This approach lays the foundation for rational selection of health promoting foods, rational target driven design of functional foods, and provides an essential thus-far, overlooked, dynamic to our understanding of how foods recognized as “healthy” impact on the human metabonome.”

Campbell J M, et al, An Enteral Formula Containing Fish Oil, Indigestible Oligosaccharides, Gum Arabic and Antioxidants Affects Plasma and Colonic Phospholipid Fatty Acid and Prostaglandin Profiles in Pigs 1, J. Nutr. 1997 127: 137-145, fed a mixture of fish oil, fructo-oligosaccharides, xylooligosacchrides, gum Arabic, and antioxidants to pigs, and reported increases in polyunsaturated fatty acid levels and decreases in the synthesis of proinflammatory prostaglandins. No difference in body weight between treated and untreated animals was reported.

Rodriguez-Cabezas M E, et al, The combination of fructooligosaccharides and resistant starch shows prebiotic additive effects in rats, Clinical Nutrition 2010: 29 832-839, used a combination of fructooligosaccharides and resistant starch in the rat to demonstrate additive prebiotic effects. The authors conclude that “functional foods based on the combination of two different dietary fibers, with different rate of fermentability along the large intestine, may result in a synergistic effect, and thus, in a more evident prebiotic effect that may confer a greater health benefit to the host.”

Cicek B, Arslan P, Kelestimur F, The Effects of Oligofructose and Polydextrose on Metabolic control Parameters in Type-2 Diabetes, Pak J Med Sci, 2009 25(4) 573-578, used a combination of oligofructose and polydextrose (FibreCal) in patients with type 2 diabetes and demonstrated improvement in glucose, lipids, and blood pressure. No comparison was made to the effects of the individual components.

Pyra, K A, Prebiotic Fibre Supplementation In Combination With Metformin Modifies Appetite, Energy Metabolism, And Gut Satiety Hormones In Obese Rats, Master's Thesis, University of Calgary, 2010, MR69600, 1-114, performed an 8-week study in the DIO rat which compared oligofructose supplementation alone, metformin alone, and the two in combination. Measurements included weight, food intake, glucose, insulin, GLP-1, PYY and other hormones measured both fasting and following an oral glucose tolerance test, and a variety of gene expression testing Statistical significance for the combination treatment as compared to either single agent was found only for (1) GIP (reduced), (2) hepatic AMPK-alpha-2 and SREBP-2 expression (both increased). The combination showed effects similar to metformin alone in terms of body weight and insulin AUC, and similar to OFS alone in terms of glucose AUC.

Hazan A, Madar Z, Preparation of a dietary fiber mixture derived from different sources and its metabolic effects in rats, J Am Coll Nutr. 1993 December; 12(6):661-8, used a mixture of apple pectin, orange pectin, locust bean gum and corncob fiber in the rat and demonstrated reductions in glycemic response, fasting cholesterol, and triglyceride concentrations. No comparisons were made to the single agents.

Hosobuchi et al, Efficacy of Acacia, Pectin and Guar Gum-Based Fiber Supplementation in the Control of Hypercholesterolemia, Nutrition Research, 1999, Vol. 19, No. 5, pp. 643-619 used a commercially-available product consisting of acacia, pectin, and guar gum in adults for four weeks and reported improvements in both total and LDL-cholesterol in treated subjects. No comparisons were made to the effects of the individual components.

Jensen, C D, Haskell W, Whittam J H, Long-Term Effects of Water-Soluble Dietary Fiber in the Management of Hypercholesterolemia in Healthy Men and Women, Am J Cardiol 1997; 79:34-37 used a mixture of psyllium, pectin, guar gum and locust bean gum for 6 months in moderately hypercholesterolemic men and women and demonstrated reductions in total and LDL cholesterol levels.

Hunninhake et al used a mixture of guar gum, pectin, soy, pea, and corn bran for 51 weeks in subjects with moderate hypercholesterolemia and demonstrated reductions in total cholesterol, LDL, and LDL/HDL ratio.

Henningsson A M, Bjorck I M, Nyman E M G L, Combinations of Indigestible Carbohydrates Affect Short-Chain Fatty Acid Formation in the Hindgut of Rats, J. Nutr. 2002 132: 3098-3104 fed guar gum and pectin separately and as a mixture to rats, and measured the short-chain fatty acid production that occurred in the hindgut. They identified that rats fed pectin had a high proportion of acetic acid in the cecum, whereas those fed guar gum had the highest proportion of proprionic acid. With the combination, the amount of butyric acid produced was twice as high as seen with the individual components. The authors conclude that “certain mixtures of indigestible carbohydrates stimulate butyric acid-producing bacteria, with potential benefits for the colonic epithelium”, and that “it remains to be elucidated whether these effects are valid also in humans and have physiologic implications for the human colonic epithelium.”

European Patent Application number 86103234.0 cites work performed using pectin or guar gum in combination with an anticholinergic medication. Various formulations were administered to individual subjects for variable periods of time. In general, weight loss and delays in gastric emptying were observed in these subjects.

In an editorial, Yarnell J W G, Evans A E, The Mediterranean diet revisited—towards resolving the (French) paradox, Q J Med 2000; 93:783-785, propose that the beneficial effects of the Mediterranean diet may be due to the combination of olive oil (high in oleic acid) and other nutritional components. They specify anthocyanins in wine as a possible contributing factor.

It has been shown in numerous clinical studies that modest weight loss (4-10%) through behavior modification (i.e. diet and exercise) markedly improves type 2 diabetes with significant reductions in hyperglycemia, dyslipidemia, and blood pressure and was associated with a significant improvement in hepatic and peripheral tissue insulin resistance. Further, studies have shown that weight loss was the most important factor in preventing diabetes from developing in patients who were deemed to be at high risk of developing type 2 diabetes. Thus, weight loss induced by a GRAS combination could facilitate anti-diabetic activity. See, for example: Kelley D E, Kuller L H, McKonalis T M, Harper P, Mancino J, Kalhan S, Effects of Moderate Weight Loss and Orlistat on Insulin Resistance, Regional Adiposity, and Fatty acids in Type 2 Diabetes, Diabetes Care 2004, 27:33-40; Knowler W C, Barrett-Connor E, Fowler S E, Hamman R F, Lachin J M, Walker E A, et al., Reduction in the incidence of type 2 diabetes with lifestyle intervention or metformi, N Engl J Med. 2002; 346(6):393-403; Sjostrom C D, Peltonen M, Wedel H, Sjostrom L, Differentiated long-term effects of intentional weight loss on diabetes and hypertension, Hypertension 36:20-25, 2000; Goldstein D, Beneficial health effects of modest weight loss. Int J Obes Relat Metab Disord 16:397-415, 1992; Wing R R, Koeske R, Epstein L H, Nowalk M P, Gooding W, Becker D, Long-term effects of modest weight loss in type II diabetic patients, Arch Intern Med 147:1749-1753, 1987; and American Diabetes Association: Evidence-based nutrition principles and recommendations for the treatment and prevention of diabetes and related complications (Position Statement), Diabetes Care 25 (Suppl. 1): S50-560, 2002.

There is increasing evidence that a range of conditions involving abnormal food intake are associated with specific patterns of gut peptides, and therefore may benefit from therapies that modulate gut peptide levels. These conditions include bulimia nervosa, which has been characterized as being associated with reduced post-prandial suppression of ghrelin and reduced levels of PYY, as detailed in Kojima et al, Altered ghrelin and PYY responses to meals in bulimia nervosa, Clinical Endocrinology, 2005:62:74-78. Anorexia nervosa is associated with increased levels of ghrelin and PYY, as characterized by Misra M, et al, Elevated peptide YY levels in adolescent girls with anorexia nervosa. J Clin Endocrinol Metab 2006; 91:1027-33. Obese individuals with and without Binge-Eating-Disorder have been shown to have reduced levels of ghrelin and PYY and a reduced post-meal PYY response, as documented in Monteleone et al, Circulating ghrelin is decreased in non-obese and obese women with binge eating disorder as well as in obese non-binge eating women, but not in patients with bulimia nervosa. Psychoneuroendocrinology 2005; 30:243-50 and Stock et al, Ghrelin, peptide YY, glucose-dependent insulinotropic polypeptide, and hunger responses to a mixed meal in anorexic, obese, and control female adolescents. J Clin Endocrinol Metab 2005; 90:2161-8. Batterham et al, demonstrate in Gut hormone PYY3-36 physiologically inhibits food intake, Nature, 2002; 418:650-654, that infusions of PYY reduce food intake in humans, which provides evidence that gut peptide modulation may be helpful in treating unrestrained food craving and food addiction. GLP-1 has well-documented effects on food intake (Gutzwiller et al, Glucagon-like peptide. 1: a potent regulator of food intake in humans, Gut 1999; 44:81-86) and oxyntomoldulin has also demonstrated beneficial effects (Cohen, et al, Oxyntomodulin Suppresses Appetite and Reduces Food Intake in Humans, J Clin Endocrinol Metab 2003; 88:4696-4701).

Syndromic excessive food intake including, but not limited to, Prader Willi Syndrome, Bardet-Biedl Syndrome: These syndromes, characterized by extreme hunger, high levels of food intake, and obesity, may benefit from modulation of gut peptides, as evidenced by initial work with a GLP-1 mimetic by Sze et al, Effects of a Single dose of Exenatide on Appetite, Gut Hormones, and Glucose Homeostasis in Adults with Prader-Willi Syndrome. J Clin Endocrinol Metab. 2011; 96(8):E1314-1319.

GLP-1-agonist therapy causes improvement in lipid parameters including reduced total and LDL cholesterol, apolipoprotein B and triglycerides, and increased HDL cholesterol, as reviewed by Davidson in Cardiovascular Effects of Glucagonlike peptide-1 Agonists, Am J Cardiol, 2011; 108(supp):33B-41B. Each of the four categories of agents described in this patent has demonstrated improvement of lipid profiles, and combinations of the agents may therefore prove efficacious as therapy for lipid abnormalities. As examples, effects of OFS and prebiotics in general are cited in Delzenne et al, Oligosaccharides: state of the art Proceedings of the Nutrition Society, 2003, 62, 177-182, in Kok et al, Insulin, GLP-1, GIP, and IGF-1 as putative mediators of the hypolipemic effect of OFS in rats Journal of Nutrition 1998 128:1099-1103, and in Ooi et al, Cholesterol-lowering effects of probiotics and prebiotics: A review of in vivo and in vitro findings International Journal of Molecular Sciences, 2010, 11: 2499-2522. The benefits of anthocyanins on lipids are cited in Tsuda, et al Regulation of Adipocyte function by anthocyanins; possibility of preventing the metabolic syndrome, J Agric Food Chem, 2008, 56, 642-646, and pectin benefits are described in Veldman et al, Dietary pectin influences fibrin network structure in hypercholesterolaemic subjects Thrombosis Research, 1997, 86(3) 183-196. The benefits of oleic acid on LDL-cholesterol and trigycerides have been well investigated, as noted by the European Food Safety Authority in their 2011 Scientific Opinion which substantiated these health claims. (EFSA Journal 2011; 9(4):2043-2060).

GLP-1 has demonstrated beneficial effects in heart failure, as demonstrated in the rat by Poornima et al, Chronic Glucagon-Like Peptide-1 Infusion Sustains Left Ventricular Systolic Function and Prolongs Survival in the Spontaneously Hypertensive, Heart Failure-Prone Rat, Circ Heart Fail 2008; 1:153-160, and in T2D patients with congestive heart failure by Nathanson et al, Effects of intravenous exenatide in type 2 diabetic patients with congestive heart failure: a double-blind, randomized controlled clinical trial of efficacy and safety, Diabetologia, 2012; 55(4):926-35. Benefits in myocardial infarction have been shown by Lonborg, et al, Exenatide reduces reperfusion injury in patients with ST-segment elevation myocardial infarction, Eur Heart J, 2012; 33(12):1491-9 and by Read et al in A pilot study to assess whether glucagon-like peptide-1 protects the heart from ischemic dysfunction and attenuates stunning after coronary balloon occlusion in humans, Circ Cardiovasc Interv, 2011; 4(3):266-72. Some early work with GLP-2 implies that this peptide may also have direct beneficial effects on cardiac tissue, as described by Penna, et al in Postconditioning with glucagon like peptide-2 reduces ischemia/reperfusion inuury in isolated rat hearts: role of survival kinases and mitochondrial KATP channels, Basic Res Cardiol, 2012; 107(4):272. Flavonols and anthocyanins have demonstrated beneficial cardiovascular effects, including protection from ischemia/reperfusion injury, as summarized by de Pascual-Teresa et al, in Flavanols and Anthocyanins in Cardiovascular Health: A Review of the Current Evidence, Int J Mol Sci, 2010; 11:1679-1703. Beneficial effects of oleic acid on various heart diseases have been well documented, as summarized by Kris-Etherton in an AHA Science Advisory: Monosaturated Fatty Acids and Risk of Cardiovascular Disease, Circulation, 1999; 100:1253-1258. Components of this invention may alter bile acid secretion and gut permeability and reduce levels of circulating lipopolysaccharide, and this may improve vascular function and heart failure (von Haehling et al. Ursodeoxycholic acid in patients with heart failure. J Am Coll Cardiol 2012; 59(6): 585-592).

In addition to the beneficial effects of weight loss on hypertension, there is evidence that the components of this invention also confer weight-loss-independent benefits. GLP-1 agonists show consistent reductions in blood pressure (Okerson, The cardiovascular effects of GLP-1 receptor agonists, Cardiovascular Therapeutics, 2012; 30:e146-155), OFS and other prebiotics have demonstrated distinct antihypertensive effects (Yeo et al, Antihypertensive Properties of Plant-Based Prebiotics, Int J of Mol Sci, 2009; 10:3517-3530), anthocyanins have been shown to reduce blood pressure and improve vascular reactivity (Jennings et al, Higher anthocyanin intake is associated with lower arterial stiffness and central blood pressure in women, Am J Clin Nutr, 2012; 96:781-8), and oleic acid also has beneficial effects (Teres et al, Oleic acid content is responsible for the reduction in blood pressure induced by olive oil, PNAS, 2008:105(37)13811-13816).

GLP-1 has been shown to be protective of vascular endothelium and capable of restoring normal endothelial permeability, as demonstrated in Dozzier et al, Glucagon-like Peptide-1 Protects Mesenteric Endothelium from Injury During Inflammation, Peptides, 2009; 30(9):1735-1741. Clinical studies using anthocyanins have shown significant improvement in a range of peripheral circulatory disorders, as cited in Fructus Myrtilli, World Health Organization Monographs on Selected Medicinal Plants, Volume 4, 2009: 217-220-221.

Metabolic Syndrome is a combination of medical conditions that, when occurring together, increase the risk for later development of diabetes, cardiovascular and cerebrovascular disease. Definitions of metabolic syndrome vary, but generally include central obesity, dyslipidemia, hypertension, and abnormalities of fasting or post-prandial glucose. Each of these conditions is addressed individually above, providing evidence of therapeutic benefit in the prevention and treatment of Metabolic Syndrome.

The cornerstone of treatment of fatty or steatotic liver disease is weight loss, and it is anticipated that therapies that demonstrate weight loss may also confer benefit in these conditions. Moderate weight loss has been shown to reverse non-alcoholic hepatic steatosis, as noted by Petersen et al in Reversal of Nonalcoholic Hepatic Steatosis, Hepatic Insulin Resistance, and Hyperglycemia by Moderate Weight Reduction in Patients with Type 2 Diabetes, Diabetes, 2005; 54(3):603-608. GLP-1 agonist therapy has been shown to reverse hepatic steatosis (Ding, et al, Exendin-4, a Glucagon-like Protein-1 (GLP-1) Receptor Agonist, Reverses Hepatic Steatosis in ob/ob Mice, Hepatology, 2006; 43(1):173-181). In addition, several of the individual components of this invention have been shown to demonstrate beneficial effects on the liver. As examples, OFS has been shown to reduce levels of liver enzymes in humans with nonalcoholic steatosis (Daubioul et al, Effects of oligofructose on glucose and lipid metabolism in patients with nonalcoholic steatohepatitis: results of a pilot study, Eur J of Clin Nut, 2005; 59: 723-726), and to reduce levels of hepatic steatosis in rats (Dauhioul, Dietary oligofructose lessens hepatic steatosis, but does not prevent hypertriglyceridemia in obese Zucker rats, J Nutr 2000; 130: 1314-1319). Specific anthocyanins have been shown to have beneficial effects on hepatic steatosis, as described by Guo et al, Cyanidin 3-glucoside attenuates obesity-associated insulin resistance and hepatic steatosis in high-fat diet-fed and db/db mice via the transcription factor Fox01, J of Nutr Biochem 2012; 23:349-360, and blackcurrant has been shown to have protective effects on the liver in chronic ethanol intoxication (Szachowicz-Petelska et al, Protective Effect of Blackcurrant on Liver Cell Membrane of Rats Intoxicated with Ethanol, J Membrane Biol, 2012; 245(4):191-200).

GLP-2 has been shown to ameliorate symptoms of inflammatory bowel disease, as discussed in Drucker et al, Human [Gly2]GLP-2 reduces the severity of colonic injury in a murine model of experimental colitis, Am J Physiol 276 (Gastro-intest Liver Physiol 39), 1999: G79-G91 and in Cani, et al, Changes in gut microbiota control inflammation in obese mice through a mechanism involving GLP-2 driven improvement of gut permeability, Gut, 2009: 58:1091-1103. Prebiotics, including OFS, have demonstrated efficacy against a range of inflammatory bowel diseases, as documented in Hedin et al, Evidence for the use of probiotics and prebiotics in inflammatory bowel disease: a review of clinical trials Proceedings of the Nutrition Society, 2007, 66, 307-315, Joossens et al, Effect of oligofructose-enriched inulin on bacterial composition and disease activity of patients with Crohn's diseas: results from a double-blinded randomized controlled trial, Gut, 2012; 61(6):958, Leenen et al, Inulin and OFS in Chronic IBD, Journal of Nutrition, 2007; 2572S-2575S, and Lomax et al, Prebiotics, immune function, infection and inflammation: a review of the evidence, British Journal of Nutrition, 2009, 101, 633-658. Pectin has been credited with reducing diarrhea, treating mouth and throat sores, minimizing radiation effects, preventing heavy-metal toxicity, and promoting ‘good digestive health’, (Sriamornsak, Chemistry of Pectin and Its Pharmaceutical Uses: A Review, Silpakorn University International Journal 2003; 3: 206-228), as well as demonstrating beneficial effects in inflammatory bowel disease (Galvez et al, Effects of dietary fiber on inflammatory bowel disease, Mol Nutr Food Res, 2005; 49(6):601-608 and Rose et al, Influence of Dietary Fiber on Inflammatory Bowel Disease and Colon Cancer: Importance of Fermentation Pattern, Nutr Rev 2007; 65(2):51-62).

Non-digestible polysaccharides, including OFS and inulin, have demonstrated therapeutic and preventative effects against a range of gastrointestinal infections, as reviewed by Gibson, et al in Prebiotics and resistance to gastrointestinal infections, British Journal of Nutrition, 2005; 93 (supp1): S31-S34. Pectin has long been used as a therapy for diarrhea, as discussed by Rabbani et al, in Clinical studies in persistent diarrhea: dietary management with green banana or pectin in Bangladeshi children, Gastroenterology, 2001: 121(3):554-60, and oleic acid has demonstrated therapeutic value for treating diarrhea via slowing of gastrointestinal transit times (Lin et al, Slowing of Gastrointestinal Transit by Oleic Acid, Digestive Diseases and Sciences, 2001:46(2):223-229).

There is increasing evidence that gut peptides play a role in immune-mediated disorders, and therefore therapies that augment gut-peptide release may be beneficial. In addition, all four categories of agents reviewed here have demonstrated beneficial immune system effects. GLP-1 therapy has demonstrated efficacy in the treatment of psoriasis (Ahern, et al, Glucagon-like peptide-1 analogue therapy for psoriasis patients with obesity and type 2 diabetes: a prospective cohort study, JEADV, 2012, DOI:10.1111/j.1468-3083.2012.04609.x, and Drucker et al, Glucagon-like peptide-1 (GLP-1) receptor agonists, obesity and psoriasis: diabetes meets dermatology, Diabetologia, 2011: 54:2741-2744). GLP-1 mimetics have been shown to reverse ER stress and apoptosis in T2D (Liang, et al, Impaired MEK Signaling and SERCA Expression Promote ER Stress and Apoptosis in Insulin-Resistant Macrophages and Are Reversed by Exenatide Treatment, Diabetes, 2012; 61(10)2609-20). GLP-2 helps to control inflammation through improvements in gut permeability (Cani, et al, Changes in gut microbiota control inflammation in obese mice through a mechanism involving GLP-2 driven improvement of gut permeability, Gut, 2009:58:1091-1103). OFS has been explored for systemic effects on atopic dermatitis, psoriasis and asthma, and for reduction of food allergies (Lomax et al, Prebiotics, immune function, infection and inflammation: a review of the evidence, British Journal of Nutrition, 2009, 101, 633-658). Blackcurrant extract has demonstrated broad antioxidant and anti-inflammatory effects (Tabart, et al, Antioxidant and anti-inflammatory activities of Ribes nigrum extracts, Food Chemistry, 2012; 131:1116-1122). Some types of pectin have also demonstrated specific anti-inflammatory effects (Silva et al, Pectin from Passiflora edulis Shows Anti-inflammatory Action as well as Hypoglycemic and Hypotriglyceridemic Properties in Diabetic Rats, J Med Food, 2011; 14(10):118-1126 and Ye et al, Dietary Pectin Regulates the Levels of Inflammatory Cytokines and Immunoglobulins in Interleukin-10 Knockout Mice, J Agric Food Chem, 2010; 58:11281-11286), and the anti-inflammatory effects of oleic acid are very well documented, as summarized by Carillo et al in Role of oleic acid in immune system: mechanism of action; a review; Nutr Hosp, 2012; 27(4)978-990. The link between inflammatory processes in the gut and risk of Type 1 Diabetes is currently under active exploration (Vaarala, Is the origin of type 1 diabetes in the gut? Immunology and Cell Biology, 2012: 90(3):271-6).

There is increasing recognition of the significant role of gut peptides in regulating bone homeostasis, and therapy that modulates gut peptides may therefore have beneficial effects on bone. Walsh, et al, in Feeding and Bone (Archives of Biochemistry and Biophysics 2010, 1:503(1), 11-9) emphasize the role of gut peptides in bone health, noting that GIP increases osteoblast number and activity and prevents PTH-induced osteoclast activation (with potentially a need for pulsatile delivery to maintain effect), that GLP-1 increases bone formation in normal rats, and that GLP-2 has demonstrated significant and acute reduction of bone resorption in a dose-dependent manner in post-menopausal women. Nuche-Berenguer, et al, have demonstrated that GLP-1 and exendin-4 can reverse hyperlipidic-related osteopenia in the rat (GLP-1 and exendin-4 can reverse hyperlipidic-related osteopenia, J of Endocrinology, 2011; 209:203-210), and clinical studies are ongoing to evaluate the effects of exendin-4 on bone in humans (ClinicalTrials.gov Identifier NCT01381926). OFS has been specifically shown to improve cation absorption (Delzenne, Oligosaccharides: state of the art, Proceedings of the Nutrition Society, 2003, 62, 177-182), and, in the ovariectomized rat, it prevents osteoporosis (Scholz-Ahrens, Effect of OFS or dietary Ca on repeated Ca and P balances, bone mineralization and trabecular structure in ovariectomized rats British Journal of Nutrition, 2002, 88, 365-377). In adolescents, OFS improves calcium absorption, which also supports bone mineralization (Van den Heuvel, et al Oligofructose stimulates calcium absorption in adolescents, Am J Clin Nutr, 1999, 69, 544-8). Anthocyanin and flavonoid intake are positively associated with bone mineral density in women (Welch, et al, Habitual flavonoid intakes are positively associated with bone mineral density in women, J Bone Miner Res, 2012: 27(9):1872-8).

GLP-1 has been shown to be beneficial in models of neurodegenerative diseases by stimulating neuronal cell proliferation, enhancing synaptic plasticity and memory formation, providing neuroprotection and decreasing neuromotor impairment, as summarized by Salcedo et al, in Neuroprotective and neurotrophic actions of glucagon-like peptide-1: an emerging opportunity to treat neurodegenerative and cerebrovascular disorders, Br J Pharmacol, 2012; 166(5):1586-99. In addition, GLP-2 has been shown to have neuroprotective effects, as cited by Voss et al in Glucagon-like peptides 1 and 2 and vasoactive intestinal peptide are neuroprotective on cultured and mast cell co-cultured rat myenteric neurons, BMC Gastroenterology 2012: 1:12-30. Anthocyanins have been shown to have neuroprotective effects, as demonstrated by Tarozzi et al, Neuroprotective effects of anthocyanins and their in vivo metabolites in SH-SY5Y cells, Neurosci Lett, 2007; 424(1)36-40, and as reviewed by Ramassamy in Emerging role of polyphenolic compounds in the treatment of neurodegenerative diseases: A review of their intracellular targets, European Journal of Pharmacology, 2006; 545:51-64.

Because of its neuroprotective effects, GLP-1 has been postulated as a potentially effective therapy for a range of disorders including bipolar disorder and major depressive disorder, as reviewed by McIntyre et al in The neuroprotective effects of GLP-1: Possible treatment for cognitive disorders, Behav Brain Res, 2012; 237C:164-171. PYY has been shown to have direct effects on mood, and its deletion enhances anxiety and depression-related behaviors, as demonstrated by Painsipp et al in The gut-mood axis: a novel role of the gut hormone peptide YY on emotional-affective behavior in mice, BMC Pharmacology, 2009(supp 2):A13. Anthocyanins have been shown to inhibit monoamine oxidases, which supports their use as potential therapies for depression, anxiety, and mood disorders. (Dreisitel et al, Berry anthocyanins and their aglycones inhibit monoamine oxidases A and B, Pharmacol Res, 2009; 59(5):306-11). An inverse relationship between dietary oleic acid intake and risk of depression has been identified in women (Wolfe, et al, Dietary linoleic and oleic fatty acids in relation to severe depressed mood: 10 years follow-up of a national cohort, Prog Neuropsychopharmacol Biol Psychiatry, 2009; 33(6):972-7).

PYY has been shown to inhibit the growth of pancreatic, esophageal, and breast cancer cells (Vona-Davis et al, PYY and the pancreas: inhibition of tumor growth and inflammation, Peptides, 2007; 28:334-338). Inulin-type fructans (OFS) reduce cancer-cell proliferation in the liver (Bindels et al, Gut microbiota-derived proprionate reduces cancer cell proliferation in the liver, British Journal of Cancer, 2012: 1-8). Inulin and OFS have demonstrated anti-cancer effects against a range of cancer types, as well as augmenting the activity of standard chemotherapeutic agents (Taper et al, Inulin/oligoructose and anti-cancer therapy, British Journal of Nutrition, 2002; 87(Supp 2):S283-S286). Pectin has been shown to have beneficial effects in prostate cancer (Jackson et al, Pectin induces aptoptosis in human prostate cancer cells: correlation of apoptotic function with pectin structure, Glycobiology, 2007; 17(8):805-819). Anthocyanins have demonstrated strong effects in preventing cancer (Wang et al, Anthocyanins and their role in cancer prevention, Cancer Lett, 2008; 269(2):281-290).

There is increasing evidence that modulation of the intestinal microbiota may have a beneficial effect on a wide range of diseases, as reviewed by Diamond et al, in It takes guts to grow a brain, Bioessays 2011; 33:588-591, by Kootte et al in The therapeutic potential of manipulating gut microbiota in obesity and type 2 diabetes mellitus, Diabetes, Obesity and Metabolism, 2012: 14:112-120, by Bravo et al in Ingestion of Lactobacillus strain regulates emotional behavior and central GABA receptor expression in a mouse via the vagus nerve, PNAS, 2011: 108(39) 16050-16055, and by Clemente et al in The Impact of the Gut Microbiota on Human Health: An Integrative View, Cell, 2012: 148:1258-1270. Several of the components of the current invention cause changes in the make-up of the gut microbiome and may therefore be of therapeutic benefit in these conditions.

GLP-1 has been shown to have beneficial effects on retinal degeneration, as would be expected because of its general neuroprotective effects, as noted by Salcedo et al, in Neuroprotective and neurotrophic actions of glucagon-like peptide-1: an emerging opportunity to treat neurodegenerative and cerebrovascular disorders, Br J Pharmacol, 2012; 166(5):1586-99. Anthocyanins have been shown to confer numerous benefits on eye health, including beneficial effects on cataracts, glaucoma, diabetic retinopathy, myopia, and night vision improvement, as summarized in Fructus Myrtilli, World Health Organization Monographs on Selected Medicinal Plants, Volume 4, 2009:217-220.

Polycystic Ovary Syndrome (PCOS): PCOS is associated with insulin resistance and hyperinsulinemia, and therapies have targeted reduced insulin resistance or increased insulin sensitivity, with agents such as metformin. Svendsen et al (Incretin hormone secretion in women with polycystic ovary syndrome: roles of obesity, insulin sensitivity, and treatment with metformin, Metabolism, 2009: 58(5):586-93) documented changes in GIP and GLP-1 in PCOS patients treated with metformin, and they conclude that this mechanism is at least partially responsible for treatment response. Therapies that modify insulin resistance and incretin levels may therefore prove beneficial.

Anthocyanins have been shown to confer significant improvement in premenstrual and dysmenorrheic symptons, as cited in Fructus Myrtilli, World Health Organization Monographs on Selected Medicinal Plants, Volume 4, 2009:220.

### BRIEF DESCRIPTION OF THE INVENTION

Briefly, in one aspect, the present invention discloses compositions comprising a pectin and an anthocyanin.

Briefly, in another aspect, the present invention discloses compositions comprising a pectin and a long-chain fatty acid.

Briefly, in another aspect, the present invention discloses compositions comprising an anthocyanin and an oligosaccharide.

Briefly, in another aspect, the present invention discloses a method of treating diabetes or obesity in a monogastric mammal comprising administration of a pectin, an anthocyanin, a long-chain fatty acid, and an oligosaccharide.

Briefly, in another aspect, the present invention discloses a method of treating diabetes or obesity in a monogastric mammal comprising administration of: GLP-1, a GLP-1 mimetic, or a drug which increases GLP-1 plasma concentration; and administration of a pectin.

Briefly, in another aspect, the present invention discloses a method of treating diabetes or obesity in a monogastric mammal comprising administration of: GLP-1, a GLP-1 mimetic, or a drug which increases GLP-1 plasma concentration; and administration of an anthocyanin; and administration of an oligosaccharide.

### DETAILED DESCRIPTION OF THE INVENTION

The compositions of the invention described above in the Brief Description, contain 2 of the following ingredients: a pectin, an anthocyanin, a long-chain fatty acid, or an oligosaccharide. Other embodiments of this invention are the above compositions comprising one more of the 4 ingredients. For example, other embodiments of this invention are:
compositions comprising an anthocyanin, an oligosaccharide, and a pectin;
compositions comprising an anthocyanin, an oligosaccharide, and a long-chain fatty acid;
compositions comprising an anthocyanin, a pectin, and a long-chain fatty acid; and
compositions comprising an oligosaccharide, a pectin, and a long-chain fatty acid.

Another embodiment of the compositions of this invention is a composition comprising a pectin, an anthocyanin, a long-chain fatty acid, and an oligosaccharide.

A method of this invention described above in the Brief Description is a method of treating diabetes or obesity in a monogastric mammal comprising administration of a pectin, an anthocyanin, a long-chain fatty acid, and an oligosaccharide. This method may optionally include co-administration of GLP-1, a GLP-1 mimetic, or a drug which increases GLP-1 plasma concentration.

A method of this invention described above in the Brief Description is a method of treating diabetes or obesity in a monogastric mammal comprising administration of: GLP-1, a GLP-1 mimetic, or a drug which increases GLP-1 plasma concentration; and administration of a pectin. This method may optionally further comprise administration of one or more of the other ingredients (an anthocyanin, an oligosaccharide, or a long-chain fatty acid). For example, other embodiments of this invention include administration of GLP-1, a GLP-1 mimetic, or a drug which increases GLP-1 plasma concentration; and administration of a pectin, and:
administration of an anthocyanan;
administration of an anthocyanin and administration of an oligosaccharide;
administration of a long-chain fatty acid;
administration of a long-chain fatty acid and administration of an anthocyanin; or
administration of a long-chain fatty acid and administration of an oligosaccharide.

A method of this invention described above in the Brief Description is a method of treating diabetes or obesity in a monogastric mammal comprising administration of: GLP-1, a GLP-1 mimetic, or a drug which increases GLP-1 plasma concentration; and administration of an anthocyanin; and administration of an oligosaccharide. This method may optionally further comprise administration of a long-chain fatty acid.

In the compositions and methods of this invention, an example of an anthocyanin is a blackcurrant extract (“BCE”).

In the compositions and methods of this invention, an example of an oligosaccharide is oligofructose.

In the compositions and methods of this invention, an example of a pectin is a pectin derived from apples.

In the compositions and methods of this invention, an example of a long-chain fatty acid is oleic acid.

In the methods of this invention, an example of a GLP-1 mimetic is an exendin-4 AlbudAb.

In one embodiment of the methods of this invention, the method is a method of treating diabetes. In another embodiment of the methods of this invention, the method is a method of treating obesity.

In the methods of this invention, an example of a monogastric mammal is a human.

Preferably, the compositions of this invention will be administered orally. It is expected that the formulations of this invention will contain some type of carrier. It is further expected that the dose for humans will be about 10 to about 40 grams per day. By 10 to 40 grams per day is meant the total amount per day of any of the four types of ingredients (anthocyanin, oligosaccharide, pectin, long-chain fatty acid). For example, if a patient were to receive 5 grams of OFS per day and 5 grams of pectin per day and 3 grams of oleic acid per day and 2 grams of BCE per day, the dose for that patient would be 15 grams per day.

The compositions of this invention include more than one of the four types of ingredients (anthocyanin, oligosaccharide, pectin, long-chain fatty acid). Preferably, the ingredient present in the least amount is present as at least 10% by weight of the highest ingredient, more preferably as at least 20% by weight of the highest ingredient.

A preferred composition of this invention comprises BCE, OFS, apple pectin, and oleic acid. In this composition, the amount of OFS in the composition is preferably from 80% to 120% by weight of the amount of apple pectin in the composition. In this composition, the amount of BCE in the composition is preferably from 20% to 60% by weight of the amount of pectin in the composition. In this composition, the amount of oleic acid in the composition is preferably from 40% to 80% by weight of the amount of pectin in the composition.

Through the same mechanisms that provide benefit for the treatment and/or prevention of Type 2 Diabetes, this invention can serve as therapy for Insulin Resistance Syndrome, Gestational Diabetes, Glucose Intolerance, and Impaired Fasting Glucose.

This invention can also provide benefit in additional therapeutic areas, some of which are listed below, because of the modulation of gut peptide levels and/or because of evidence of direct treatment benefit conferred by one or more components of this invention:
Non-syndromic abnormal food intake including, but not limited to, binge eating, anorexia, unrestrained food craving, food addiction;
Syndromic excessive food intake including, but not limited to, Prader Willi Syndrome, Bardet-Biedl Syndrome;
Dyslipidemia, including, but not limited to, fasting and post-prandial dyslipidemia, hyperlipidemia, hypercholesterolemia, hypertriglyceridemia;
Heart Failure and Myocardial Infarction;
Hypertension;
   Peripheral Circulatory Disorders, including but not limited to peripheral arteriovascular disease, varicose veins, telangiectasis, peripheral vascular insufficiency, and diabetogenic peripheral edema;
Metabolic Syndrome;
Liver diseases including, but not limited to, hepatic steatosis, non-alcoholic fatty liver disease, non-alcoholic steatohepatitis, hepatic fibrosis, hepatic insufficiency, alcoholic liver disease, and post-liver-transplant therapies:
Gastrointestinal disorders including, but not limited to irritable bowel syndrome, Crohn's disease, ulcerative colitis, short bowel syndrome, constipation, inflammatory bowel disease, celiac disease;
Infectious diarrheas and post-infectious diarrhea syndromes including but not limited to post-antibiotic colitis;
Immune-mediated disorders, including but not limited to celiac disease, asthma, psoriasis, eczema, rheumatoid arthritis, ankylosing spondylitis, Type 1 Diabetes, Latent Autoimmune Diabetes of Adults;
Bone mineralization disorders, osteoporosis, osteopenia;
Neurodegenerative diseases, including, but not limited to Parkinson's syndrome, Alzheimer's disease, post-traumatic syndromes, Huntington's disease, and Amyotrophic Lateral Sclerosis, post-cerebrovascular accident recovery;
Psychiatric Disorders, including but not limited to depression, schizophrenia, bipolar disorder, mood disorders, anxiety disorder;
Cancer Treatment and Prevention;
Diseases and conditions that may benefit through modulation of the gut microbiome, including but not limited to obesity, type 2 diabetes, type 1 diabetes, immune-mediated diseases, neurodevelopmental diseases, cancer, asthma and respiratory conditions, gastrointestinal disorders, cognition, and emotional behavior;
Diseases of the eye, including but not limited to macular degeneration, retinal degeneration, cataracts, glaucoma, diabetic retinopathy, myopia, inflammatory disorders of the eye, presbyopia and therapy to improve night vision;
Polycystic Ovary Syndrome (PCOS);
Premenstrual Syndrome and Dysmenorrhea.

### Examples

A variety of compositions comprising one or more of an anthocyanin (BCE), an oligosaccharide (OFS), a pectin (apple pectin), and a long-chain fatty acid (oleic acid) were evaluated. Each of these components is found in small amounts in a normal diet, and each has GRAS (Generally Recognized As Safe) status for use as a food ingredient. All combinations were evaluated both with and without co-administration of exendin-4 AlbudAb (a GLP-1 mimetic). Since there are 4 types of ingredients, there are 4 singles, 6 pairs, 4 triplets, and one of all four for a total of 15 combinations of one or more of the 4 types of ingredients. The following examples demonstrate that for some of these compositions, when administered together in the Diet-induced obesity (DIO) mouse (a model of obesity) or the db/db mouse (a model of diabetes) along with drugs such as a GLP-1 mimetic (e.g. exendin-4 AlbudAb), the weight loss and/or glucose lowering observed far exceeds the sum of the effects seen with each individual GRAS components.

In the following experiments, The BCE was purchased from Cyvex Nutrition, Irvine, Calif. (lot #1734-018); the OFS was purchased from Beneo Inc., Morris Plains, N.J. (lot #PCRNX9BNX9); the apple pectin was purchased from Herbstreith&Fox, Elmsford, N.Y. (Classic AU201 USP lot 01104302); the oleic acid was purchased from Sigma, St. Louis, Mo. (lot # MKBD0534V); and the Nutella was purchased from Ferrero, Somerset, N.J. (lot #34RT236B3 and 37R258C3).

In the following experiments, the GLP-1 mimetic used for co-administration was an exendin-4 AlbudAb, a long-acting GLP-1 mimetic. This molecule has been used as a representative of the GLP-1 class due to its extended half-life in rodent species as compared to currently available marketed agents. The exendin-4 AlbudAb is a recombinant fusion protein consisting of exendin-4 genetically fused to a variable domain of the light chain of an antibody. Exendin-4 is currently marketed as exenatide. The AlbudAb is a domain antibody consisting of a small (˜14 kDa) human antibody light chain variable domain that binds to human serum albumin. Following subcutaneous injection, the AlbudAb portion of the molecule binds to endogenous albumin, leading to a significant increase in half-life (t_{1/3}). In rodents, the AlbudAbs have t_{1/2}=20-40 hrs, as compared to the 4 to 6 hour half-life of the unadorned peptide counterpart, AlbudAb is a trademark of the GlaxoSmithKline group of companies. The exendin-4-albudAb was prepared by dilution into vehicle, 20 mM Sodium Citrate+100 mM NaCl, pH 6.2 and frozen at −70° C. in aliquots. On each day of dosing, aliquots were thawed just prior to dosing and maintained on ice.

### Chronic Obesity Efficacy Studies:

Male diet induced obese (DIO) C57BL/6 mice (40-50 g body weight) and lean C57BL/6 mice (Taconic, Hudson, N.Y.) were used for the chronic obesity efficacy studies. The DIO mice were group housed and fed a high fat diet (60% fat by kcal) by the vendor from the time of weaning. Upon receipt at the GlaxoSmithKline Research Triangle Park facility, all mice were single-housed and maintained at constant temperature (approximately 22° C.) with 12 hr light/dark cycle (lights on from 5:00 AM to 5:00 PM). The mice were given ad libitum access to food (Research Diets D12451, 45 kcal % fat for DIO; Lab Diet 5001, 13.5 kcal % fat for lean) and water. All animal protocols were approved by the institutional animal care and use committee at GlaxoSmithKline in Research Triangle Park, N.C.

The BCE, OFS, pectin, oleic acid, Nutella containing chows were prepared by mixing in a Hobart mixer with a whisk attachment and stored at 4° C. until used. Chows were fed to mice in glass jars with wire mesh column inserts (Unifab Corp., Kalamazoo, Mich.).

DIO C57BL/6 mice and age-matched lean controls were habituated in house for approximately 4 weeks before the start of the study. The mice were randomized into treatment groups with similar mean body weights. The mice were acclimated to 25% Nutella (w/w) in Research Diets D12451 meal chow for 5 days, and then fed 2% w/w BCE, 5% w/w OFS, 5% w/w pectin, and 3% w/w oleic acid in the 25% Nutella in D12451 chow alone and in all combinations of 2, 3 and 4 for 34 days (beginning on day −7). All combination then were evaluated with and without co-treatment with exendin-4-albudAb. The various treatments are summarized in Table 1 below. Chows were replaced approximately weekly.

TABLE 1

| Group | | Ex4 |
|---|---|---|
| No. | Treatment | AlbudAb |
| 1 | Vehicle | |
| 2 | 5% OFS | |
| 3 | 5% Pectin | |
| 4 | 2% BCE | |
| 5 | 3% Oleic Acid | |
| 6 | 2% BCE + 5% OFS | |
| 7 | 2% BCE + 5% Pectin | |
| 8 | 2% BCE + 3% Oleic Acid | |
| 9 | 5% OFS + 5% Pectin | |
| 10 | 5% OFS + 3% Oleic Acid | |
| 11 | 5% Pectin + 3% Oleic Acid | |
| 12 | 2% BCE + 5% OFS + 5% Pectin | |
| 13 | 2% BCE + 5% OFS + 3% Oleic Acid | |
| 14 | 2% BCE + 5% Pectin + 3% Oleic Acid | |
| 15 | 5% OFS + 5% Pectin + | |
| | 3% Oleic Acid | |
| 16 | 2% BCE + 5% OFS + 5% | |
| | Pectin + 3% Oleic Acid | |
| 17 | Ex4 AlbudAb (0.03 mg/kg) | (+) |
| 18 | 5% OFS | (+) |
| 19 | 5% Pectin | (+) |
| 20 | 2% BCE | (+) |
| 21 | 3% Oleic Acid | (+) |
| 22 | 2% BCE + 5% OFS | (+) |
| 23 | 2% BCE + 5% Pectin | (+) |
| 24 | 2% BCE + 3% Oleic Acid | (+) |
| 25 | 5% OFS + 5% Pectin | (+) |
| 26 | 5% OFS + 3% Oleic Acid | (+) |
| 27 | 5% Pectin + 3% Oleic Acid | (+) |
| 28 | 2% BCE + 5% OFS + 5% Pectin | (+) |
| 29 | 2% BCE + 5% OFS + 3% Oleic Acid | (+) |
| 30 | 2% BCE + 5% Pectin + | (+) |
| | 3% Oleic Acid | |
| 31 | 5% OFS + 5% Pectin + | (+) |
| | 3% Oleic Acid | |
| 32 | 2% BCE + 5% OFS + 5% | (+) |
| | Pectin + 3% Oleic Acid | |

On day −1, the mice were dosed subcutaneously with vehicle to acclimate them to handling stress. The exendin-4 AlbudAb fusion was dosed subcutaneously every second day (e.o.d.) between 2-4 pm with a dose volume of 5 ml/kg over a period of 26 days (day 0 to 26; 14 doses). Mice not receiving exendin-4 AlbudAb were dosed with vehicle.

Baseline consumption of the 25% Nutella chow was established during the 5 day acclimation period (day −12 to −7); daily food intake measurements were taken on week days beginning on day −7. Body weights (BW) were measured on day −7 and then every 3 to 4 days for the duration of the study. On day 24, body composition was measured using quantitative magnetic resonance (QMR). On day 27, approximately 19 hours after the last dose of exendin-4 AlbudAb, whole blood samples were collected under isoflurane anesthesia and processed to serum and plasma. Serum was used to evaluate clinical chemistry parameters (e.g. glucose, etc.).

Table 2 summarizes the range of weight-loss data seen with various combinations and highlights the degree of variability and the efficacy achieved with GLP-1 co-administration.

TABLE 2

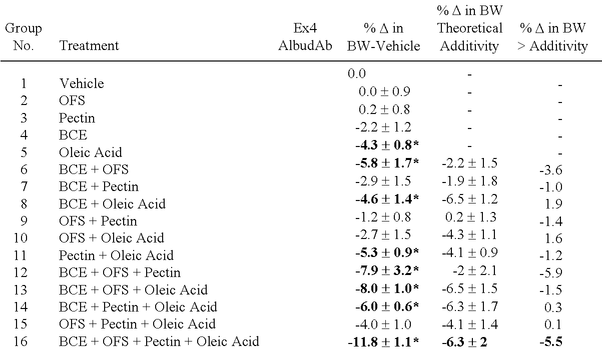

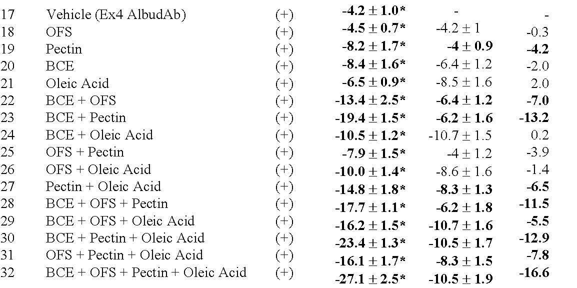

*Denotes statistical significance, % Δ in BW vs. Vehicle; p<0.05, Student's t-test.
Grey cell color denotes significance, %> Δ in BW additivity vs. % Δ in BW theoretical additivity; p<0.05, Student's t-test.
N = 8 except for the following groups: (1) BCE + Pectin, N = 7; (2) BCE + Oleic Acid, N = 7; (3) BCE + OFS + Oleic Acid, N = 7; BCE + Pectin + Ex4 AlbudAb, N = 7; BCE + OFS + Pectin + Ex4 AlbudAb, N = 6

The data in Table 2 show that most of the single agents, with (groups 18-21) or without co-administration of GLP-1 mimetic (groups 2-5), had little to no effect on weight compared to vehicle. Among those without co-administration of GLP-1 mimetic, only oleic acid (group 5) had statistically significant weight loss compared to vehicle. Among those with co-administration of GLP-1 mimetic, only pectin (group 19) had unexpected weight loss compared to pectin alone and GLP-1 mimetic alone. In addition, the GLP-1 mimetic also had statistically significant weight loss compared to vehicle. As the various ingredients are combined, or co-administered in the case of GLP-1 mimetic, some agents that performed well alone turned out to be antagonistic when co-administered, resulting in no net effect on weight. Other agents paired extremely well together, demonstrating weight loss that significantly exceeded the additive effect of the individual agents.

Looking at the 6 pairs with (groups 22-27) or without (groups 6-11) co-administration of GLP-1 mimetic, only 3 pairs, each of which was with co-administration of GLP-1 mimetic, had unexpected weight loss compared to their individual ingredients alone and GLP-1 mimetic alone. These 3 pairs are BCE+ pectin (group 22), BCE+ oleic acid (group 23) and pectin+ oleic acid (group 27)

Looking at the 4 triplets with (groups 28-31) or without (groups 12-15) co-administration of GLP-1 mimetic, only the triplets co-administered with GLP-1 mimetic had unexpected weight loss compared to their individual ingredients alone and GLP-1 mimetic alone.

The composition containing an example of each of the four ingredients both with (group 32) and without (group 16) co-administration of GLP-1 mimetic had unexpected weight loss compared to the individual ingredients alone and GLP-1 mimetic alone. The combination of OFS, pectin, BCE, and oleic acid (Group 16) resulted in weight loss of −11.8% in 26 days, far exceeding what would be expected based on the weight loss of OFS, pectin, BCE, and oleic acid when administered alone (0%, 0.2%, −2.2% and −4.3%, respectively, with a projected additive weight loss of −6.3%; resulting in a −5.5% weight loss > additivity; P<0.05). Group 32 which comprises OFS, pectin, BCE, and oleic acid in combination with the exendin-4 AlbudAb (ED₂₀ dose for weight loss; 0.03 mg/kg) resulted in weight loss of −27.1% in 26 days, far exceeding what would be expected based on the weight loss of exendin-4 AlbudAb and OFS, pectin, BCE, and oleic acid when administered alone (−4.2% and 0%, 0.2%, −2.2%, −4.3%, respectively, with a projected additive weight loss of −10.5%; resulting in a −16.6% weight loss > additivity; P<0.05).

In summary the compositions of groups 16, 19, 22, 23, 27, 28, 29, 30, 31, and 32 each had unexpected results regarding weight loss. Furthermore, the Group 32 treatment resulted in normalization of body weight, body composition and clinical chemistry parameters (e.g. glucose, cholesterol, triglycerides, AST, ALT) back to lean control values. In addition groups 19, 22, 23, 27, 28, 29, 30, and 31 also resulted in statistically significant reductions in body composition and various clinical chemistry parameters (e.g. varying changes in either glucose, cholesterol, triglycerides, AST and/or ALT) commensurate with the body weight loss.

Chronic Diabetes Efficacy Studies:

Db/db mice (B6.Cg-m+/+Lepr db/J), (40-50 g body weight) and age-matched controls were habituated in house for approximately 4 weeks before the start of the study. Body weight and body composition was measured and the mice were randomized into treatment groups with similar mean % body fat and body weight.

The mice were acclimated to 25% Nutella (w/w) in Lab Diet chow 5K67 meal chow, (16 kcal % fat) for 11 days, and then fed 1.3% w/w BCE, 3.3% w/w OFS, 3.3% w/w pectin, and 2% w/w oleic acid in the 25% Nutella 5K67 chow. On day −1, the mice were dosed subcutaneously with vehicle to acclimate them to handling stress. The exendin-4 AlbudAb fusion was dosed subcutaneously every second day (e.o.d.) between 2-4 pm with a dose volume of 5 ml/kg over a period of 15 days (day 0 to 14; 8 doses). Mice not receiving exendin-4 AlbudAb were dosed with vehicle.

Baseline consumption of the 25% Nutella chow was established during the 11 day acclimation period (day −18 to −7); daily food intake measurements were taken on week days beginning on day −7. Body weights were measured on day −7 and then every 3 to 4 days for the duration of the study. On day 14, body composition was measured using quantitative magnetic resonance (QMR). On day 15, approximately 19 hours after the last dose of exendin-4 AlbudAb, whole blood samples were collected under isoflurane anesthesia and processed to serum and plasma. Whole blood was used to determine % HbA1c. Serum was used to evaluate clinical chemistry parameters (e.g. glucose, etc.).

In db/db mice, 14-day administration of OFS, pectin, BCE, oleic acid and exendin-4 AlbudAb (ED₂₀ dose) in combination significantly reduced glucose (Δ−217 mg/dL; p<0.001) and HbA1c levels (Δ−1.2%; p<0.001) relative to vehicle control. Further, the reduction in glucose (−217 mg/dL vs. −142 mg/dL expected additivity) and HbA1c (−1.2% vs. −0.7% expected additivity) with the combination being more than the expected sum of each component. The weight loss/inhibition of weight gain was more than additive for the OFS, pectin, BCE, oleic acid and exendin-4 AlbudAb combination in the db/db mice (−7.4% vs. −3.8% expected additivity; P<0.05). The combination in the db/db mice resulted in statistically significant changes in lipid parameters such as triglycerides (Δ−53% from vehicle; p<0.05) and cholesterol (Δ−34% from vehicle; p<0.05) as well as liver enzymes such as alanine aminotransferase (Δ−48% from vehicle; p<0.05).

Chronic Obesity Efficacy Study Exemplifying Another GLP-1 Mimetic (Liraglutide):

A chronic (20-day) in vivo efficacy study was conducted in DIO C57BL/6 mice, similarly to what was described previously with the Exendin-4 albudAb, to investigate the efficacy of OFS, pectin, BCE, oleic acid in combination with another long-acting GLP-1 analog (liraglutide) in reducing body weight and improving metabolic parameters. Liraglutide (Victoza) is an analog of human GLP-1 classified as a GLP-1 receptor agonist, developed as a treatment for Type 2 Diabetes. Treatment with liraglutide results in clinically relevant lowering of HbA1c along with dose dependent weight-loss in diabetic subjects.

In DIO C57BL/6 mice, 20 day administration of 1.7% w/w BCE, 3.3% w/w OFS, 3.3% w/w pectin, and 2% w/w oleic acid in chow and liraglutide (0.02 mg/kg, ED₂₀ dose for weight loss) subcutaneously; n=8/group) in combination yielded weight loss that trended toward the unexpected more than additive weight loss that was obtained with the combination of BCE, OFS, pectin and oleic acid with exendin-4 AlbudAb. In this study mice were treated with liraglutide first for 6 days followed by combination with OFS, pectin, BCE, oleic acid for 15 days (20 days total). Mice treated with OFS, pectin, BCE, oleic acid for 15 days lost −10.6% bodyweight. Treatment with liraglutide alone for 20 days resulted in a final weight loss of −5.4% while mice treated with OFS, pectin, BCE, oleic acid+ liraglutide lost −20.1%, which exceeded the projected additivity of −16.0%. Similar changes in body composition, serum chemistries and hormone analytes were observed with the liraglutide+OFS, pectin, BCE, oleic acid combination as were seen with the exendin-4 AlbudAb combination that correlated to the amount of weight loss changes.

What is claimed is:

1. A composition comprising a pectin and an anthocyanin.

2. The composition of claim 1 further comprising an oligosaccharide.

3. A composition comprising a pectin and a long-chain fatty acid.

4. The composition of claim 3 further comprising an anthocyanin.

5. The composition of claim 3 further comprising an oligosaccharide.

6. A composition comprising an anthocyanin and an oligosaccharide.

7. The composition of claim 6 further comprising a long-chain fatty acid.

8. The composition of claim 7 further comprising a pectin.

9. The composition of any of claims 1, 2, 4, 6, 7, or 8 wherein said anthocyanin is derived from blackcurrant.

10. The composition of any of claims 2, 5, 6, 7, or 8 wherein said oligosaccharide is oligofructose.

11. The composition of any of claims 1-5 or 8 wherein said pectin is derived from apples.

12. The composition of any of claims 3, 4, 5, 7, or 8 wherein said long-chain fatty acid is oleic acid.

13. A method of treating diabetes or obesity in a monogastric mammal comprising administration of a pectin, an anthocyanin, an oligosaccharide, and a long-chain fatty acid.

14. The method of claim 13 further comprising administration of GLP-1, a GLP-1 mimetic, or a drug which results in an increase in GLP-1 plasma concentration.

15. A method of treating diabetes or obesity in a monogastric mammal comprising administration of:
GLP-1, a GLP-1 mimetic, or a drug which results in an increase in GLP-1 plasma concentration;
and a pectin.

16. The method of claim 15 further comprising administration of an anthocyanin.

17. The method of claim 16 further comprising administration of an oligosaccharide.

18. The method of claim 15 further comprising administration of a long-chain fatty acid.

19. The method of claim 18 further comprising administration of an anthocyanin.

20. The method of claim 18 further comprising administration of an oligosaccharide.

21. A method of treating diabetes or obesity in a monogastric mammal comprising administration of:
GLP-1, a GLP-1 mimetic, or a drug which results in an increase in GLP-1 plasma concentration;
an anthocyanin; and
and an oligosaccharide.

22. The method of claim 21 further comprising administration of a long-chain fatty acid.

23. The method of any of claims 13, 14, 16, 17, 19, 21, or 22 wherein said anthocyanin is derived from blackcurrant.

24. The method of any of claims 13, 14, 17, 20, 21, or 22 wherein said oligosaccharide is oligofructose.

25. The method of any of claims 13-20 wherein said pectin is derived from apples.

26. The method of any of claims 13, 14, 18-20, or 22 wherein said long-chain fatty acid is oleic acid.

27. The method of any of claims 14-26 wherein said GLP-1 mimetic is an exendin-4 AlbudAb.

28. The method of any of claims 14-26 wherein said drug which results in an increase in GLP-1 plasma concentration is metformin or a DPPIV inhibitor.

29. The composition of any of claims 1-12 wherein the claimed ingredient that is present in the least amount is present as at least 10% by weight of the claimed ingredient that is present in the highest amount.

30. The composition of any of claims 1-12 wherein the claimed ingredient that is present in the least amount is present as at least 20% by weight of the claimed ingredient that is present in the highest amount.

31. The composition of claim 8 wherein said anthocyanin is BCE, said oligosaccharide is oligofructose, said pectin is an apple pectin, and said long-chain fatty acid is oleic acid.

32. The composition of claim 31 wherein the amount of oligofructose in the composition is from 80% to 120% by weight of the amount of the apple pectin in the composition, the amount of BCE in the composition is from 20% to 60% by weight of the amount of the apple pectin in the composition, and the amount of oleic acid in the composition is from 40% to 80% by weight of the amount of the apple pectin in the composition.
